(19) United States
(12) Patent Application Publication
(10) Pub. No.: US 2022/0193367 A1
(43) Pub. Date: Jun. 23, 2022

(54) STEERABLE CATHETER DESIGN WITH SPINE-REINFORCED MOLDED ARTICULATION JOINT

(71) Applicant: Boston Scientific Scimed Inc., Maple Grove, MN (US)

(72) Inventors: David Benjamin Joda, Minneapolis, MN (US); Raymond David Gessler, III, Roberts, WI (US); Poorva Milind Rajguru, Shoreview, MN (US); Emily Rose Whitwam, Blaine, MN (US); Brian Paul Edison, Otsego, MN (US); Colin Patrick Dunphy, St Louis Park, MN (US); Joseph Nelsen, Forest Lake, MN (US); Brian Soltis, St Paul, MN (US)

(21) Appl. No.: 17/558,777

(22) Filed: Dec. 22, 2021

Related U.S. Application Data
(60) Provisional application No. 63/129,960, filed on Dec. 23, 2020.

(51) Int. Cl. A61M 25/00; A61M 25/01

(52) U.S. Cl. CPC A61M 25/0053; A61B 18/1492; A61M 25/0147

(57) ABSTRACT

An electrophysiology catheter comprising a tubular shaft, the shaft having a proximal portion, a distal portion having a distal end and a deflection region, the shaft defining a longitudinal axis and including an outer tubular jacket, and articulation member disposed within the jacket in the deflection region, the articulation member comprising a plurality of longitudinally-arranged tubular segments, a plurality of first connecting segments, and a plurality of second connecting segments, wherein adjacent tubular segments are joined by respective ones of the first and second connecting segments, and wherein all of the first and second connecting segments are disposed in a first plane extending through the longitudinal axis. A first reinforcing member and a second reinforcing member extend through the plurality of first and second connecting segments, respectively.

### CROSS REFERENCE TO RELATED APPLICATION

This application claims priority to Provisional Application No. 63/129,960, filed Dec. 23, 2020, which is herein incorporated by reference in its entirety.

### TECHNICAL FIELD

The present invention relates to medical devices and methods for catheters for medical procedures. More specifically, the invention relates to devices and methods that include directional enhancement for catheters such as steerable catheters.

### BACKGROUND

Various medical procedures involve catheters inserted into a patient's vasculature. In certain procedures, the catheter may be navigated through the vasculature to a target location in the body. The distal end of the catheters may be inserted into the patient's heart chambers in, for example, interventional electrophysiology procedures. The distal end of the catheter may include one or more electrodes that are used to delivery therapy (e.g., ablation) or map the surface of the heart tissue (e.g., identify the locations of heart tissue that are a source of the arrhythmias). Accurately locating and maintaining a location of the catheter, including the distal end portion of the catheter, may facilitate catheter performance.

### SUMMARY

In Example 1, an electrophysiology catheter comprising a tubular shaft having a proximal portion, a distal portion having a distal end and a deflection region. The shaft defines a longitudinal axis and includes an outer tubular jacket, an articulation member disposed within the jacket in the deflection region, and first and second reinforcing members. The articulation member comprises a plurality of longitudinally-arranged tubular segments, a plurality of first connecting segments, and a plurality of second connecting segments, wherein respective ones of the first and second connecting segments are disposed between and join adjacent tubular segments, and wherein the first and second connecting segments are disposed in a first plane extending through the longitudinal axis. The first reinforcing member and the second reinforcing member extend. through the plurality of first and second connecting segments, respectively.

In Example 2, the electrophysiology catheter of Example 1, wherein the first and second connecting segments are configured as living hinges integrally formed with the tubular segments.

In Example 3, the electrophysiology catheter of either of Examples 1 or 2, wherein the first and second reinforcing members are embedded, respectively, within the first and second connecting segments and within the tubular segments.

In Example 4, the electrophysiology catheter of any of Examples 1-3, wherein the first and second reinforcing members each comprise a helically wound flat ribbon wire.

In Example 5, the electrophysiology catheter of any of Examples 1-3, wherein the first and second reinforcing members are stranded wire cables.

In Example 6, the electrophysiology catheter of any of Examples 1-5, wherein the first and second reinforcing members are embedded within the tubular segments through direct overmold to form a bond with the first and second connecting members, and wherein the bond is further strengthened through adhesives or surface treatments.

In Example 7, the electrophysiology catheter of any of Examples 1-6, wherein the deflection region is configured to assume a curved shape when a deflection force is applied to the distal portion of the shaft, wherein the curved shape lies in a second plane through the longitudinal axis, the second plane being orthogonal to the first plane.

In Example 8, the electrophysiology catheter of Example 7, wherein the first and second reinforcing members are configured to maintain the curved shape substantially aligned with the second plane in response to the deflection force.

In Example 9, the electrophysiology catheter of any of Examples 1-8, wherein the first and second reinforcing members are configured to resist deflection of the distal portion of the shaft out of the second plane.

In Example 10, the electrophysiology catheter of any of Examples 1-9, further comprising a handle attached to the proximal end of the tubular shaft, the handle including a steering actuator, and a first steering wire extending through the tubular shaft, the first steering wire being operatively coupled to the steering actuator and anchored to the distal portion of the tubular shaft, wherein actuation of the steering actuator causes the first steering wire to apply the deflection force to the distal portion of the tubular shaft so as to cause the deflection region to assume the curved shape.

In Example 11, the electrophysiology catheter of Example 10, wherein the first steering wire is circumferentially offset from the first and second reinforcing members by 90 degrees.

In Example 12, the electrophysiology catheter of either of Examples 10 or 11, further comprising a second steering wire extending through the shaft, the second steering wire being operatively coupled to the steering actuator and anchored to the distal portion of the shaft, the second steering wire being circumferentially offset from the first steering wire by 180 degrees such that the first and second steering wires lie in the second plane.

In Example 13, the electrophysiology catheter of any of Examples 7-12, further comprising a first steering wire lumen and a second steering wire lumen within the plurality of tubular segments around the first and second steering wires.

In Example 14, the electrophysiology catheter of any of Examples 1-13, further comprising one or more ablation electrodes disposed at the distal end of the tubular shaft.

In Example 15, the electrophysiology catheter of any of Examples 1-14, wherein the tubular segments further comprises a center lumen, and wherein the outer tubular jacket further comprises an outer layer and a braided middle layer.

In Example 16, an electrophysiology catheter comprising a tubular shaft having a proximal portion, a distal portion having a distal end and a deflection region. The shaft defines a longitudinal axis and includes an outer tubular jacket, an articulation member, and first and second reinforcing members. The articulation member is disposed within the jacket in the deflection region and comprises a plurality of longitudinally-arranged tubular segments, a plurality of first connecting segments, and a plurality of second connecting segments, wherein adjacent tubular segments are joined by respective ones of the first and second connecting segments, and wherein all of the first and second connecting segments are disposed in a first plane extending through the longitudinal axis. The first reinforcing member and the second reinforcing member extend through the plurality of first and second connecting segments, respectively.

In Example 17, the electrophysiology catheter of Example 16, wherein the first and second reinforcing members are embedded, respectively, within the first and second connecting segments and within the tubular segments.

In Example 18, the electrophysiology catheter of Example 17, wherein the first and second reinforcing members each comprise a helically wound flat ribbon wire.

In Example 19, the electrophysiology catheter of Example 17, wherein the first and second reinforcing members are stranded wire cables.

In Example 20, the electrophysiology catheter of Example 17, wherein the first and second reinforcing members are embedded within the tubular segments through direct overmold to form a bond with the first and second connecting members.

In Example 21, the electrophysiology catheter of Example 16, wherein the deflection region is configured to assume a curved shape when a deflection force is applied to the distal portion of the shaft, wherein the curved shape lies in a second plane through the longitudinal axis, the second plane being orthogonal to the first plane.

In Example 22, the electrophysiology catheter of Example 21, wherein the first and second reinforcing members are configured to maintain the curved shape substantially aligned with the second plane in response to the deflection force.

In Example 23, the electrophysiology catheter of Example 21, further comprising first and second steering wire lumens extending through the tubular segments, wherein each steering wire lumen is configured to receive a steering wire configured to apply the deflection force and cause the deflection section to assume the curved shape, wherein the first and second steering wires are circumferentially offset from the first and second reinforcing members by about 90 degrees:

In Example 24, an electrophysiology catheter comprising a tubular shaft having a proximal portion, a distal portion having a distal end and a deflection region. The shaft defines a longitudinal axis and includes an outer tubular jacket, an articulation member disposed within the jacket in the deflection region, and first and second reinforcing members. The articulation member comprises a plurality of longitudinally-arranged tubular segments, and a plurality of living hinges, wherein adjacent tubular segments are joined by a respective pair of living hinges, and wherein all of the living hinges are disposed in a first plane extending through the longitudinal axis. The first and second reinforcing members extend through the plurality of living hinges.

In Example 25, the electrophysiology catheter of Example 24, wherein the first and second reinforcing members are embedded, respectively, within the living hinges and within the tubular segments.

In Example 26, the electrophysiology catheter of Example 25, wherein the first and second reinforcing members each comprise a helically wound flat ribbon wire.

In Example 27, the electrophysiology catheter of Example 26, wherein the first and second reinforcing members each comprise a stiff polymer or metal material.

In Example 28, the electrophysiology catheter of Example 26, wherein the first and second reinforcing members are embedded within the tubular segments through direct overmold to form a bond with the first and second connecting members.

In Example 29, the electrophysiology catheter of Example 26, wherein the deflection region is configured to assume a curved shape when a deflection force is applied to the distal portion of the shaft, wherein the curved shape lies in a second plane through the longitudinal axis, the second plane being orthogonal to the first plane.

In Example 30, the electrophysiology catheter of Example 29, wherein the first and second reinforcing members are configured to maintain the curved shape substantially aligned with the second plane in response to the deflection force.

In Example 31, the electrophysiology catheter of Example 29, further comprising a first steering wire lumen and a second steering wire lumen within the plurality of tubular segments and configured to receive respective steering wires for applying the deflection force, wherein the first and second steering wire lumens are each circumferentially offset from the first and second reinforcing members by about 90 degrees.

In Example 32, an electrophysiology catheter comprising a tubular shaft having a proximal portion, a distal portion having a distal end and a deflection region. The shaft defines a longitudinal axis and includes an outer tubular jacket, an articulation member disposed within the jacket in the deflection region, and a first reinforcing member and a second reinforcing member. The articulation member comprises a plurality of connected, longitudinally-arranged tubular segments. The first reinforcing member and the second reinforcing member extend longitudinally through the tubular segments in a first plane extending through the longitudinal axis.

In Example 33, the electrophysiology catheter of any of Examples 32, wherein the first and second reinforcing members each comprise a helically wound flat ribbon wire.

In Example 34, the electrophysiology catheter of any of Examples 33, wherein the first and second reinforcing members are stranded wire cables.

In Example 35, the electrophysiology catheter of any of Examples 33, wherein the first and second reinforcing members are embedded within the tubular segments through direct overmold to form a bond with the first and second connecting members, and wherein the bond is further strengthened through adhesives or surface treatments.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is an illustration of an example electrophysiology catheter, consistent with various aspects of the present disclosure.

FIG. 2 is an illustration of the electrophysiology catheter, shown in FIG. 1, as deflected in a first direction, consistent with various aspects of the present disclosure.

FIG. 3 is an illustration of the electrophysiology catheter, shown in FIGS. 1-2, as deflected in a second direction, consistent with various aspects of the present disclosure.

FIG. 4 is a side-view illustration of a portion of a shaft of the electrophysiology catheter including a deflection region, consistent with various aspects of the present disclosure.

FIG. 5A is a top-view illustration of an articulation member of the electrophysiology catheter, consistent with various aspects of the present disclosure.

FIG. 5B is a cross-sectional illustration of a tubular segment of the electrophysiology catheter, consistent with various aspects of the present disclosure.

FIG. 5C is a side-view illustration of an articulation member of the electrophysiology catheter, consistent with various aspects of the present disclosure.

FIG. 5D is a detailed illustration of a portion of a side-view illustration of an articulation member of the electrophysiology catheter, consistent with various aspects of the present disclosure.

FIG. 6 is another illustration of an example electrophysiology catheter, consistent with various aspects of the present disclosure.

FIG. 7A is an embodiment of a side-view illustration of a portion of an articulation member of the electrophysiology catheter, consistent with various aspects of the present disclosure.

FIG. 7B is an embodiment of a side-view illustration of a portion of an alternative embodiment of a articulation member of the electrophysiology catheter, consistent with various aspects of the present disclosure.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

Various aspects of the present disclosure are directed toward directional enhancement of catheters and steerable catheters. When arranged within a patient, catheters and steerable catheters may be steered or curved in a number of directions. The present disclosure includes aspects that facilitate and enhance directionality of the catheters and steerable catheters. As described in further detail below, at least portion of the catheters and steerable catheters may include directional enhancement features that bias the catheters and steerable catheters in a desired direction when curved.

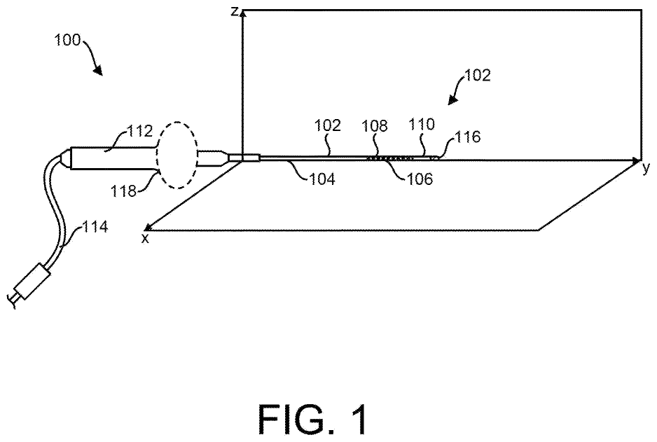

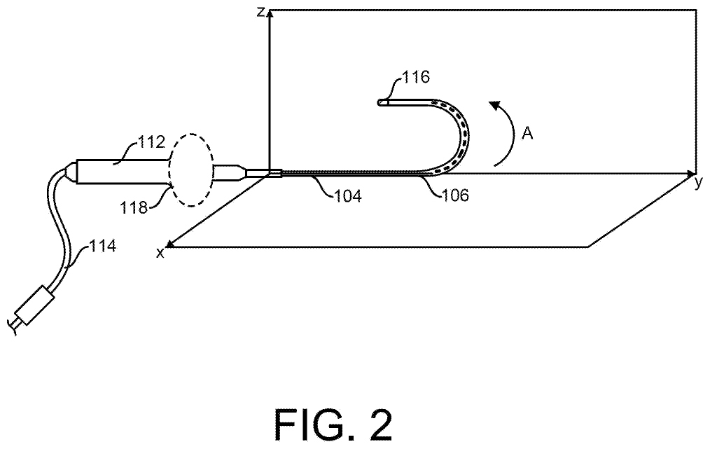

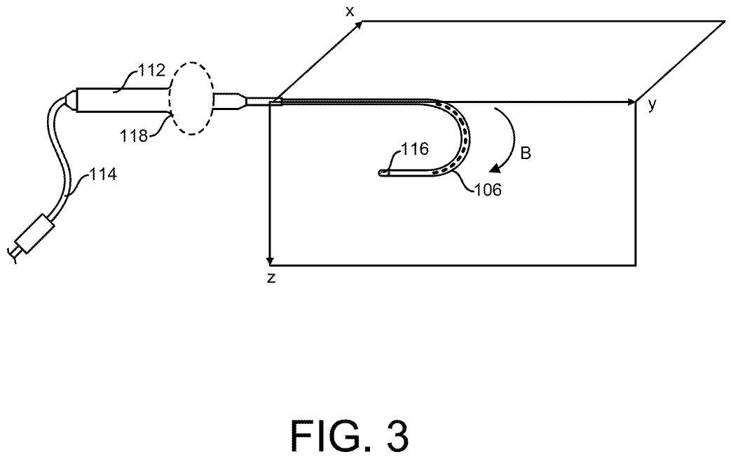

FIGS. 1-3 are illustrations of an example electrophysiology catheter 100, consistent with various aspects of the present disclosure. As shown in FIG. 1, the electrophysiology catheter 100 may be a steerable electrophysiology catheter 100. In certain instances, the electrophysiology catheter 100 is steerable in one direction (e.g., direction A as shown in FIG. 2) or in multiple directions (e.g., directions A and B as shown in FIGS. 2-3). The electrophysiology catheter 100 generally includes a tubular shaft 102 having a proximal portion 104 and a distal portion 106 that is sized and configured for placement and manipulation within in a target area of a heart of patient. The distal portion 106 may be steerable. As shown, the distal portion 106 further includes a deflection region 108 and a distal end 110.

The tubular shaft 102, in an undeflected state, defines a longitudinal axis, which may generally correspond to the geometrical centerline of the tubular shaft 102. In addition, in an undeflected state, the tubular shaft 102 may define mutually perpendicular planes (e.g. cartesian planes) extending through and intersecting along the longitudinal axis of the tubular shaft 102. In one embodiment, as shown in FIG. 1, the mutually perpendicular planes are represented by the axes x-y and y-z, with the tubular shaft 102 defining the axis y.

In certain instances, the tubular shaft 102 extends from a distal portion of a handle 112. An electrical cable or other suitable connector 114 extending from a proximal end of the handle 112 may be coupled to a source of energy or other equipment (not shown in FIG. 1) for transmitting one or more ablation signals. FIG. 1 generally illustrates one or more ablation electrodes 116 disposed at the distal end 110 of the distal portion 106.

A steering actuator 118, such as a rotatable knob or plunger that may be arranged at a distal end of the handle 112, may be manipulated by a physician to deflect or position the steerable distal portion 106 of the tubular shaft 102.

As shown in FIGS. 2-3, the electrophysiology catheter 100 is of the deflectable or steerable type, such that during use, the deflection region 108 can be deflected or curved by a user to facilitate locating the distal end 110 and the ablation electrodes 116 at a desired target location within the heart. In embodiments, deflection of the deflection region 108 can be accomplished by manipulation of the steering actuator 118, which is operatively connected to steering elements (e.g., wires, tendons, ribbons, and the like) extending within and attached (directly or indirectly) to the shaft 102 at a location within the distal portion 106. The particular mode and structure for deflecting the deflection region 108 is not critical to the present disclosure, and so any technique, whether now known or later developed, can be employed within the scope of the present disclosure.

In use, deflecting or curving the distal portion 106 may impart a torsional force that could torque or twist the distal portion 106 away from or out of the target location. The deflection region 108 may torque out of plane from the plane in which the distal portion 106 was arranged prior to deflection due to the tension on the curvature of the electrophysiology catheter 100 within vasculature. As will be discussed in more details below, the catheter 100 is configured such that the planarity of the deflection region 108 is maintained substantially along the y-z plane.

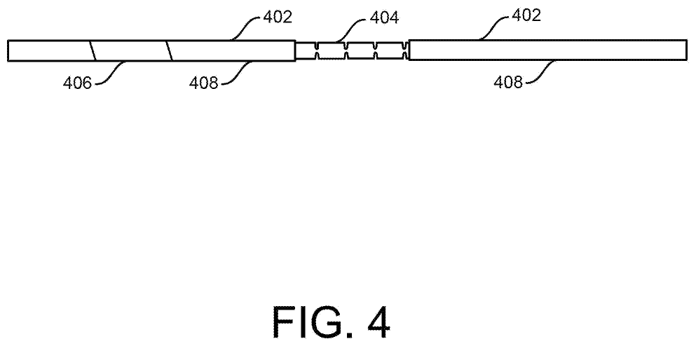

FIG. 4 is side view of a portion of the shaft 102 including the deflection region 108, consistent with various aspects of the present disclosure. As shown, shaft 102 includes an outer tubular jacket 402 (shown partially cut away in FIG. 4), and an articulation member 404 disposed within the outer tubular jacket 402.

In certain instances, the outer tubular jacket 402 is of a braided construction having a polymer material reinforced with a metal or polymeric braid formed of a plurality of interwoven wires that are woven, knitted, entwined or otherwise interlaced together. The skilled artisan will recognize that the use of braided jackets in catheter construction is well known, and the particular details of the braid/jacket construction are not of critical importance to the present disclosure. In embodiments, the aforementioned braid can be omitted, or alternatively, other constructions, e.g., reinforcing coils, can be employed to enhance the structural and torsional strength of the jacket 402.

As will be explained in greater detail below, and with reference to FIGS. 2-3, the articulation member 404 is configured to exhibit a relatively high degree of flexibility in the y-z plane, while at the same time being relatively inflexible in the x-y plane. As such, the articulation member 404 facilitates predictable, highly planar deflection of the deflection region 108 by resisting torsional forces on the shaft 102 that would otherwise tend to cause the deflection region 108 to deflect or bend in the x-y plane (or some other plane oriented transversely to the y-z plane).

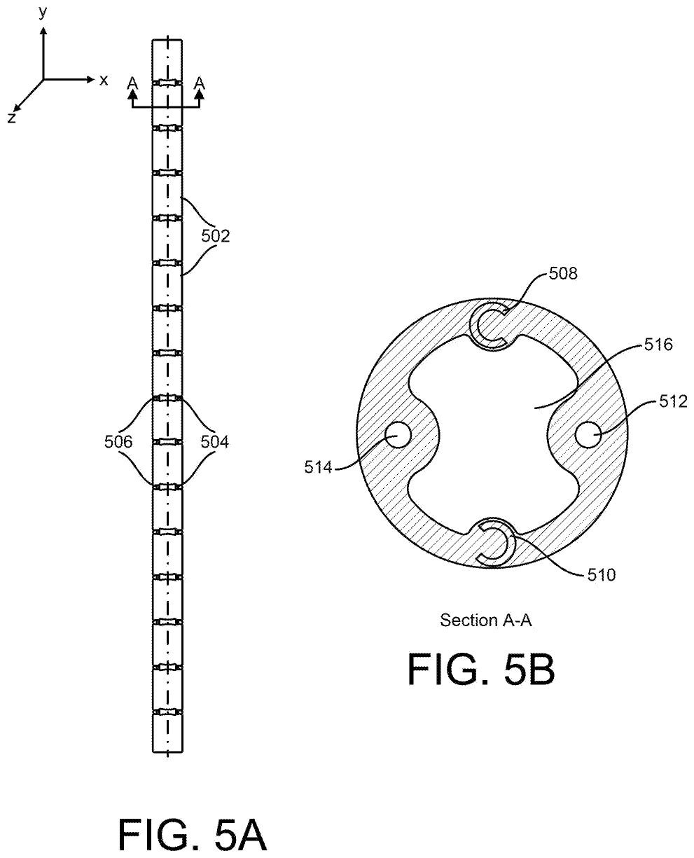

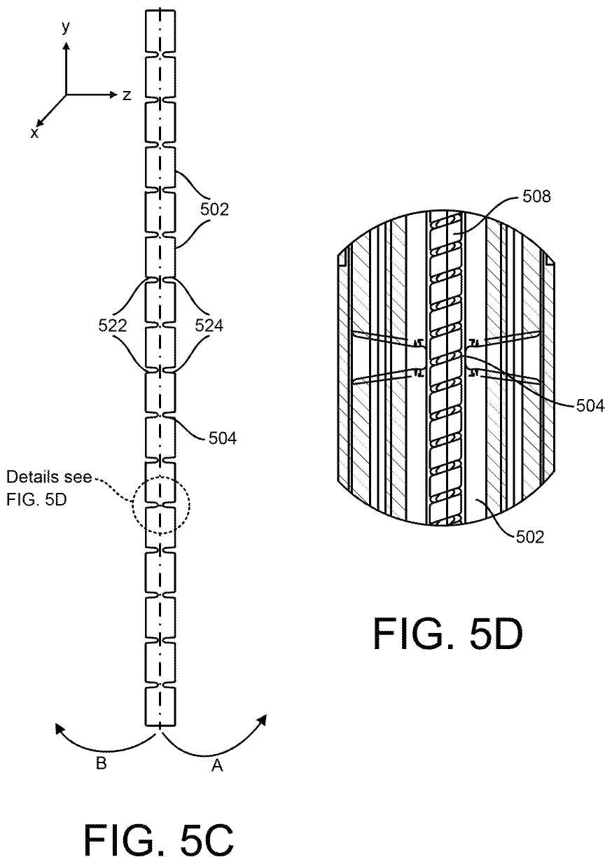

FIG. 5A is a top-view illustration of an articulation member 404 of the electrophysiology catheter 100, consistent with various aspects of the present disclosure. As shown, the articulation member 404 includes a plurality of longitudinally arranged tubular segments 502, a plurality of connecting segments 504, and a plurality of connecting segments 506. The adjacent tubular segments 502 are joined by respective ones of the connecting segments 504, 506. As shown, the connecting segments 504, 506 are disposed in the x-y plane when the articulation member 404 is in an undeflected state, represented by the cartesian coordinate system shown along with FIG. 5A.

FIG. 5B is a cross-sectional illustration of the tubular segments 502 of the electrophysiology catheter 100 taken along the line A-A in FIG. 5A, consistent with various aspects of the present disclosure. As shown, the articulation member 404 includes a pair of reinforcing members 508, 510 extending therethrough. As further shown, the reinforcing members 508, 510 are embedded within the connecting segments 504, 506, respectively, to strengthen the connections between adjacent tubular segments 502, as will be discussed in further detail below.

As shown, the tubular segments 502 may include a pair of steering wire lumens 512, 514 arranged in a plane defined by axes y and z, perpendicular to the x-y plane in which the connecting segments 504, 506 lie. As one of skilled in the art would appreciate, the steering wire lumens 512, 514 are configured to receive steering wires or members that are operatively connected to the steering actuator 118 (FIG. 1) to facilitate the deflection of at least the deflection region 108 of the tubular shaft 102 as known in the conventional manner. In some embodiments, the segments 502 may include only one steering wire lumen.

As shown, the steering wire lumens 512, 514 (and consequently, the steering wires received within each, respectively) are located at diametrically opposite positions from one another about the circumference of the articulation member 404. As such, the steering wire lumens 512, 514 are circumferentially offset from one another by about 180 degrees, and are each circumferentially offset from the reinforcing members 508, 510 by about 90 degrees. In this configuration, the steering wires lumens 512, 514 lie in the y-z plane.

FIG. 5C is a side-view illustration of the articulation member 404 of the electrophysiology catheter 100, consistent with various aspects of the present disclosure. As shown, the tubular segments 502 are connected through the connecting segments 504, 506 disposed in the plane defined by axes x and y, represented by the cartesian coordinate system shown along with FIG. 5C. The articulation member 404 shown in FIG. 5C is 90 degrees offset from the articulation member 404 shown in FIG. 5A, which is also demonstrated by the rotation of the x and z axes in the two figures.

The connecting segments 504, 506 function as living hinges integrally formed with the tubular segments 502. In response to a deflection force, the articulation member 404 may bend in a direction A along the y-z plane. In certain embodiments, the articulation member 404 may bend in another direction B, opposite to the direction A along the y-z plane. Whether the articulation member 404 is unidirectional or bidirectional is not of critical importance to the current invention.

Regardless of which direction (e.g., A or B) the articulation member 404 bends, the living hinges create a plurality of slits 522 and a plurality of slits 524 in between each adjacent tubular segments 502 so as to articulate the bending to be substantially within the y-z plane. In some embodiments, the slits 522, 524 between different adjacent tubular segments 502 are equal. In other embodiments, the plurality of slits 522, 524 between different adjacent tubular segments 502 can be different to fine-tune articulation of the distal portion 106 of the tubular shaft 102. In some embodiments, the slits may be concave and substantially “V-shaped,” or substantially “U-shaped.”

The living hinge design enables the articulation member 404 to exhibit a relatively high degree of flexibility in the y-z plane, while at the same time being relatively inflexible in the x-y plane (the plane passing through the living hinges). As such, the plurality of slits 522, 524 facilitate the articulation member 404 to have predictable, highly planar deflection of the deflection region 108 by resisting torsional forces on the shaft 102 that would otherwise tend to cause the deflection region 108 to deflect or bend in the x-y plane (or some other plane oriented transversely to the y-z plane).

FIG. 5D is a detailed illustration of a portion of a side-view illustration of the articulation member 404 of the electrophysiology catheter 100, consistent with various aspects of the present disclosure. As shown, the reinforcing member 508 is embedded within the connecting segments 504, and within the tubular segments 502.

In embodiments, the materials used for the living hinges can be relatively soft to minimize the potential for stress-induced failures of the connecting segments 504, 506. Softer material deforms easily during articulating, thus limiting ability for fine movements and decreasing overall durability. Therefore, the reinforcing members 508, 510 are made of stiffer materials than the connecting segments 504, 506. In some embodiments, the reinforcing members 508, 510 each includes a helically round flat ribbon wire. In certain instances, the reinforcing members 508, 510 may include coils, stranded wire cables, stiffer polymer materials, and other metal or polymer components. In other instances, the materials used for the reinforcing members 508, 510 can be altered according to the specific needs of the application.

In embodiment, the tubular segments 502 may include a center lumen 516 arranged centrally within the tubular segments 502 along the tubular shaft 102. In certain instances, the center lumen 516 may include components such as one or more wires for ablation electrodes arranged along the tubular shaft 102, navigational components, a temperature sensor (e.g., thermocouple), a force sensor, radio-frequency circuitry and/or wires, and a cooling lumen. The center lumen 516 may be a working channel through which one or more devices may be passed.

As shown, the center lumen 516 may be of an hourglass shape. In some embodiments, the center lumen 516 may be substantially of a round or circular shape. The pair of steering wire lumens 512, 514 may be arranged on opposite sides of the center lumen 516.

In certain instances, the steering wire lumens 512, 514 may extend along the y-z plane and may be aligned with the x-y plane. The steering wire lumens 512, 514 are configured to receive steering elements (e.g., wires, tendons, ribbons made of stainless steel, titanium, MP35N, a suitable alloy, and other suitable materials) to apply deflection forces to deflect or curve at least the deflection region 108 of the tubular shaft 102.

The deflection force may twist or torque the deflection region 108 out of approximately the y-z plane due to tension on the steering wires lumens 512, 514 and/or the curvature of the electrophysiology catheter 100 within vasculature. The connecting segments 504, 506 are configured to maintain planarity of the deflection region 108 in response to curvature of the deflection region 108. The connecting segments 504, 506 may stabilize the deflection region 108. For example, the connecting segments 504, 506 may create a bias at the deflection region 108 to substantially align with the deflection. In certain instances, the connecting segments 504, 506 may resist bending out of the y-z plane. The connecting segments 504, 506 are configured to maintain the deflection region 108 approximately within the y-z plane (e.g., within approximately +/−10% of the plane) while the deflection force curve or deflect at least the distal portion 106 of the tubular shaft 102. In certain instances, the connecting segments 504, 506 are configured to maintain the deflection region 108 substantially aligned (e.g., within approximately +1-10%) with the y-z plane in response to the deflection force.

The connecting segments 504, 506 may maintain planarity of the deflection region 108 and lessen reliance of bending planarity on the relatively less stiff (e.g., polymer materials) of the tubular segments 502 of the tubular shaft 102. The connecting segments 504, 506 may maintain a curved shape of the deflection region 108 during deflection and maintain repeatability of achieving the curved shape of the deflection region 108 during deflection.

In embodiments, the plurality of tubular segments 502 may be substantially of the same length. In other embodiments, the length of the tubular segments 502 may be varied to customize or fine tune the deflection shape of at least the deflection region 108 of the tubular shaft 102.

In some instances, the reinforcing members 508, 510 are embedded within the tubular segments 502 through direct overmold to form a bond between the tubular segments 502 and the connecting members 504, 506. The bond between the tubular segments 502 and the connecting members 504, 506 may be further strengthened through adhesives, surface treatments, or other applications that may enhance the bond.

Direct overmolding allows for high-strength joints between the adjacent tubular segments 502 that still maintain desired flexibility. In some embodiments, the reinforcing members 508, 510 have higher surface area due to the helically round flat ribbon shape to allow for stronger mechanical bond or lamination into the molded connecting segments 504, 506.

Thus, the plurality of tubular segments 502 may be made of stiffer materials to increase durability and articulation feedback during use. For instance, the tubular segments 502 may be made of polycarbonate (PC), acrylonitrile butadiene styrene (ABS), PC/ABS blends, polyetheretherketone (PEEK), acetal, polyetherimide, or liquid crystal polymer (LCP).

The direct overmolding process greatly decrease processing time and operator interaction compared to stringing adjacent tubular segments 502 over the connecting segments 504, 506 separately. This process allows lower inherent variation by creating the entire articulation member 404 in one process. The mold can be sized to allow the reinforcing members 508, 510 to pass along length of part while preventing significant flow of material through the connecting segments 504, 506. The constraints at the connecting segments 504, 506 allow them to be molded with or without tension. In addition, tab or oval-shaped gates are used at parting line to allow flow through and/or around the reinforcing members 508, 510, thus achieving proper containment.

The reinforcing members 508, 510 prevent compression during articulation, leading to greater consistency after higher cycle counts.

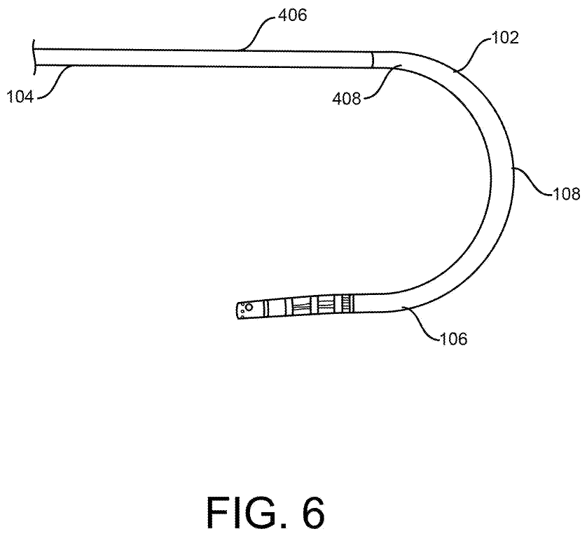

FIG. 6 is another illustration of an example electrophysiology catheter 100, consistent with various aspects of the present disclosure. As discussed in detail above, the electrophysiology catheter 100 includes a tubular shaft 102 having a deflection region 108. The tubular shaft 102 includes an outer tubular jacket 402. In some embodiments, the outer tubular jacket 402 may include multiple layers (e.g. an outer layer made of TPE, TPU such as PEBA, or other material with a durometer less than 40D, and a braided middle layer of high coverage, high pick-per-inch (PPI), multi-wire braid).

The articulation member 404 (shown in FIG. 5A-D) is disposed within the deflection region 108. The articulation member 404 facilitate maintaining planarity of the curvature of the deflection region 108. For example, the articulation member 404 is configured to create a force perpendicular to the deflection (e.g., caused by the deflection force) creating a bend between the pair of reinforcing members 508, 510.

The articulation member 404 being arranged within the deflection region 108 of the tubular shaft 102 operates to stabilize at least the distal portion 106 of the tubular shaft 102. In other instances, the articulation member 404 may extend along a length of the tubular shaft 102 or extend into an intermediate section between the proximal portion 104 and the distal portion 106.

The articulation member 404 embedded within the deflection region 108 of the tubular shaft 102 may be caused to assume a curved shape when the tubular shaft 102 is arranged within a patient's vasculature. The ability to selectively curve the deflection region 108 can facilitate accurate and effective delivery therapy (e.g., ablation) or map the surface of the heart tissue (e.g., identify the locations of heart tissue that are a source of the arrhythmias).

In embodiments, the braided structure can operate to reinforce the connecting segments 504, 506 during torsional or tensile loading. The combination of high PPI braid for a middle layer and low durometer material for an outer layer may increase elasticity of the outer tubular jacket 402. The high elasticity of the outer tubular jacket 402 prevents undue stress during articulation by reducing compression while stretching the outer tubular jacket 402. However, the use of braided jackets in catheter construction is well known in the art, and the particular details of the braid/jacket construction are not of critical importance to the present disclosure. In embodiments, the aforementioned braid can be omitted, or alternatively, other constructions, e.g., reinforcing coils, can be employed to enhance the structural and torsional strength of the jacket 402.

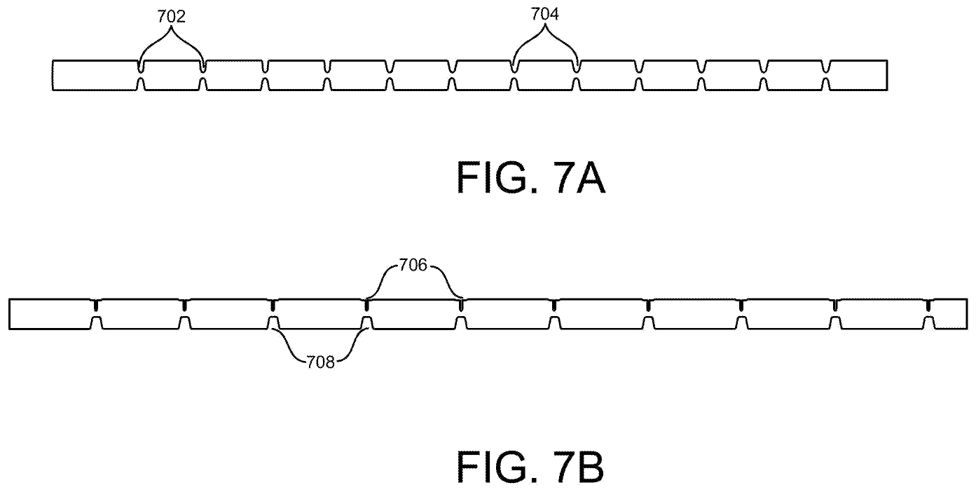

FIG. 7A is an embodiment of a side-view illustration of a portion of the articulation member 404 according to another embodiment of the disclosure. As shown, the plurality of slits 702 are of a different shape than the plurality of slits 704 so as to allow different bend radii in the regions of the articulation member 404 where each of the plurality of slits are located. For example, in the illustrated embodiment, the slits 702 are narrower than the slits 704. As a result, when a deflection force is applied so as to cause the articulation member 404 to bend, the bend radius of the portion of the articulation member 404 containing the slits 702 will be larger than the bend radius of the portion containing the slits 704. Accordingly, selectively providing slits 702, 704 of different widths along different portions of the articulation member 404 can allow the articulation member 404 achieve multi-radius curvatures.

FIG. 7B is an embodiment of a side-view illustration of a portion of an alternative embodiment of the articulation member 404 that facilitates assumption of unidirectional or asymmetric curves in the articulation member 404. As shown, the plurality of slits 706 are substantially closed gaps, e.g., the opposing tubular segment faces spanned by the slits 706 are in contact, or spaced very closely, when the articulation member 404 is in its undeflected state. As further shown, the slits 708 are relatively wide. In this embodiment, the articulation member 404 is substantially inflexible in the direction of bending toward the slits 706, while still allowing the articulation member 404 to bend in the direction of the slits 708. The articulation member 404 as shown in this figure would be relatively straight and undeflected in the direction towards the side where the narrow slits 706 are, so as to achieve substantially unidirectional bending. In other embodiments, the slits 706 and 708 can be selectively located along the articulation member 404 so as to allow the articulation member 404 to assume asymmetric bending profiles, e.g., wherein the deflection point in the bending direction toward the slits 706 is different than the deflection point in the bending direction toward the slits 708. The skilled artisan will readily appreciate that still different configurations of the articulation member 404 and the slits 706, 708 can be employed to further tune the bending profile as desired.

In other embodiments, slits of different cut width, depth, shape, and locations on either side of the articulation member 404 can be changed to fine tune the curve characteristics, thus creating asymmetry in the bending or articulation.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. An electrophysiology catheter comprising:
a tubular shaft having a proximal portion, a distal portion having a distal end and a deflection region, the shaft defining a longitudinal axis and including:
an outer tubular jacket;
an articulation member disposed within the jacket in the deflection region, the articulation member comprising a plurality of longitudinally-arranged tubular segments, a plurality of first connecting segments, and a plurality of second connecting segments, wherein adjacent tubular segments are joined by respective ones of the first and second connecting segments, and wherein all of the first and second connecting segments are disposed in a first plane extending through the longitudinal axis; and
a first reinforcing member and a second reinforcing member extending through the plurality of first and second connecting segments, respectively.

2. The electrophysiology catheter of claim 1, wherein the first and second reinforcing members are embedded, respectively, within the first and second connecting segments and within the tubular segments.

3. The electrophysiology catheter of claim 2, wherein the first and second reinforcing members each comprise a helically wound flat ribbon wire.

4. The electrophysiology catheter of claim 2, wherein the first and second reinforcing members are stranded wire cables.

5. The electrophysiology catheter of claim 2, wherein the first and second reinforcing members are embedded within the tubular segments through direct overmold to form a bond with the first and second connecting members.

6. The electrophysiology catheter of claim 1, wherein the deflection region is configured to assume a curved shape when a deflection force is applied to the distal portion of the shaft, wherein the curved shape lies in a second plane through the longitudinal axis, the second plane being orthogonal to the first plane.

7. The electrophysiology catheter of claim 6, wherein the first and second reinforcing members are configured to maintain the curved shape substantially aligned with the second plane in response to the deflection force.

8. The electrophysiology catheter of claim 6, further comprising first and second steering wire lumens extending through the tubular segments, wherein each steering wire lumen is configured to receive a steering wire configured to apply the deflection force and cause the deflection section to assume the curved shape, wherein the first and second steering wires are circumferentially offset from the first and second reinforcing members by about 90 degrees:

9. An electrophysiology catheter comprising:
a tubular shaft having a proximal portion, a distal portion having a distal end and a deflection region, the shaft defining a longitudinal axis and including:
an outer tubular jacket;
an articulation member disposed within the jacket in the deflection region, the articulation member comprising a plurality of longitudinally-arranged tubular segments, and a plurality of living hinges, wherein adjacent tubular segments are joined by a respective pair of living hinges, and wherein all of the living hinges are disposed in a first plane extending through the longitudinal axis; and
a first reinforcing member and a second reinforcing member extending through the plurality of living hinges.

10. The electrophysiology catheter of claim 9, wherein the first and second reinforcing members are embedded, respectively, within the living hinges and within the tubular segments.

11. The electrophysiology catheter of claim 10, wherein the first and second reinforcing members each comprise a helically wound flat ribbon wire.

12. The electrophysiology catheter of claim 11, wherein the first and second reinforcing members each comprise a stiff polymer or metal material.

13. The electrophysiology catheter of claim 11, wherein the first and second reinforcing members are embedded within the tubular segments through direct overmold to form a bond with the first and second connecting members.

14. The electrophysiology catheter of claim 11, wherein the deflection region is configured to assume a curved shape when a deflection force is applied to the distal portion of the shaft, wherein the curved shape lies in a second plane through the longitudinal axis, the second plane being orthogonal to the first plane.

15. The electrophysiology catheter of claim 14, wherein the first and second reinforcing members are configured to maintain the curved shape substantially aligned with the second plane in response to the deflection force.

16. The electrophysiology catheter of claim 14, further comprising a first steering wire lumen and a second steering wire lumen within the plurality of tubular segments and configured to receive respective steering wires for applying the deflection force, wherein the first and second steering wire lumens are each circumferentially offset from the first and second reinforcing members by about 90 degrees.

17. An electrophysiology catheter comprising:
a tubular shaft having a proximal portion, a distal portion having a distal end and a deflection region, the shaft defining a longitudinal axis and including:
an outer tubular jacket;
an articulation member disposed within the jacket in the deflection region, the articulation member comprising a plurality of longitudinally-arranged tubular segments; and
a first reinforcing member and a second reinforcing member extending longitudinally through the tubular segments in a first plane extending through the longitudinal axis.

18. The electrophysiology catheter of any of claim 17, wherein the first and second reinforcing members each comprise a helically wound flat ribbon wire.

19. The electrophysiology catheter of any of claim 18, wherein the first and second reinforcing members are stranded wire cables.

20. The electrophysiology catheter of any of claim 18,
wherein the first and second reinforcing members are embedded within the tubular segments through direct overmold to form a bond with the first and second connecting members; and
wherein the bond is further strengthened through adhesives or surface treatments.
